(19) United States
(12) Patent Application Publication
(10) Pub. No.: US 2022/0356434 A1
(43) Pub. Date: Nov. 10, 2022

(54) MAGNETIC CELL CARRIER COMBINED WITH A POWERLESS BIOREACTOR SYSTEM TO CELL AMPLIFICATION KIT

(71) Applicant: National Health Research Institutes, Miaoli County (TW)

(72) Inventors: FENG-HUEI LIN, Miaoli County (TW); CHING-YUN CHEN, Miaoli County (TW); YUN-YI WANG, Miaoli County (TW); YUN-MING WANG, Miaoli County (TW)

(21) Appl. No.: 17/382,028

(22) Filed: Jul. 21, 2021

(30) Foreign Application Priority Data
May 4, 2021 (TW) 110116063

(51) Int. Cl. C12M 1/42; C12M 3/06; C12M 1/00; C12N 1/04; G01N 33/49

(52) U.S. Cl. CPC C12M 35/06; C12M 23/16; C12M 39/00; C12N 1/04; G01N 33/49; B82Y 5/00

(57) ABSTRACT

A magnetic cell biocarrier combined with a powerless bioreactor system comprising a biocarrier, a powerless bioreactor, and a magnetic field device. The biocarrier can detach the cells through temperature regulation and can be adsorbed by the magnetic field device to stabilize at the bottom of the gooseneck cell culture tank; the powerless bioreactor comprises a microinfusion element, a culture fluid collection element, and a gooseneck cell culture tank; the internal space of the gooseneck cell culture tank is interconnected with the microinfusion element and the culture fluid collection element, the microinfusion element slowly injects fresh culture medium When the culture medium in the gooseneck cell culture tank is above an overflow position, the cell metabolites can be automatically discharged to the culture fluid collection element by the interconnected vessels to reduce the risk of cell contamination.

### FIELD OF INVENTION

The present invention relates to a bioreactor system for culturing cells, especially a bioreactor system for stably amplifying cultured cells, and the function and shape of the cultured cells are closer to the cells in the human body and applied to the regenerative medicine field.

### BACKGROUND OF THE INVENTION

In recent years, as the regenerative medicine market increases yearly and relevant laws and regulations are revised by various countries, the demand for stem cells as cell therapy materials has increased markedly.

There are the biggest challenges of cell therapy for building a good culture system that can maintain the characteristics and functions of stem cells in the cell culture scale-up process. Therefore, there are limited techniques for culturing a large number of stem cells in vitro.

Although the conventional two-dimensional culture can proliferate cells, not only can it not reconstruct the function and shape of a stem cell similar to that in vivo due to the flat and rigid two-dimensional environment, but it also costs considerable labor and laboratory costs. Conventional technology has developed biomedical materials using bioreactors with cell scaffolds or microcarriers in three-dimensional (3D) cell culture. The biomedical materials can be used to mimic the extracellular matrix (ECM) network structure and function in vitro, and the mechanical strength of the carrier can also be adjusted to meet the needs of cells.

The cell culture method of the conventional bioreactor refers to a tissue culture system in which cells and small cell clusters are cultured in a liquid culture medium that is constantly stirred or shaken, wherein the cell clusters need to be attached to the biological culture medium for culture. However, the biological culture media are mostly chemically synthesized substances, which may induce cell differentiation. Instead, even culture medium cannot automatically replace the cell suspension culture, it is the best culture method in the current state of the art.

Furthermore, there are two commonly biological carriers used in the cell suspension culture model of three-dimensional culture; one is an artificial scaffold with interior porous, and the other is a microstructure with a diameter of about 130-380 μm. Regarding the artificial scaffold, it requires a long immersing time in trypsin or carrier-decomposing solution during cell recovery, which will affect cell viability and damage its ECMs. Regarding the microstructure, it may drift within the culture fluid in the perfusion bioreactor due to low density and then may block the culture system eventually.

In addition, in the prior art, cells are cultured under the stimulation of static magnetic fields of different intensities, which can help cell viability and promote proliferation.

Accordingly, a need is identified for a bioreactor that addresses the limitations of the culture medium cannot be automatically replaced, the biological carrier would block the culture system, and low recovery rate of conventional bioreactors.

### SUMMARY OF THE INVENTION

An object of the present invention is to solve the above-mentioned problems of the prior art, and provides a magnetic cell carrier combined with a powerless bioreactor system for culturing cells, comprising: a cell carrier, a powerless bioreactor, and a magnetic field device; wherein,

the cell carrier has a porous and wrinkling surface to enhance the ability for cell attachment; the interior of the cell carrier is a three-dimensional porous structure, which enables large surface area and space; the cell carrier also exhibits temperature-responsive cell adhesion/detachment properties;

the powerless bioreactor comprises a micro-infusion element, a culture fluid collection element, and a gooseneck cell culture tank, wherein the micro-infusion element is responsible for importing fresh culture solution into the gooseneck cell culture tank; wherein culture fluid collection element is used for recovering the culture medium containing cell metabolites discharged from the gooseneck cell culture tank; the gooseneck cell culture tank comprises a cover and a bottle, wherein the cover comprises a first hole and a second hole, and a first hole comprises air outlet tube and an air inlet tube, wherein the air inlet tube is used for supplying oxygen to the cells, and the air outlet tube is used to discharge the carbon dioxide released by the cells; the bottle body comprises a gooseneck tube which is used for draining the culture medium containing cell metabolites to the culture fluid collection element; and,

the magnetic field device is used for attracting the cell carrier.

In an embodiment wherein said the gooseneck cell culture tank is connecting with the micro-infusion element and the culture fluid collection element, and the micro-infusion element can slowly import fresh culture medium through the infusion tube to the gooseneck cell culture tank; when the culture medium in the gooseneck cell culture tank is higher than an overflow position L, the medium containing cell metabolites can be automatically discharged through the gooseneck tube to the culture fluid collection element by the principle of communicating vessels to reduces the risk that the culture medium might be contaminated during culture; the nutrient in the gooseneck cell culture tank adopted a semi-replacement condition to simulate the dynamic environment in which nutrients and metabolites coexist in the body;

In another embodiment wherein said the cell carrier of the present invention is a natural biopolymers gelatin mixed with magnetic nanoparticles, and a chemical cross-linking agent is used to enhance the mechanical properties and strength amino group of the gelatin. Another water-soluble cross-linking agent carbodiimide (EDC) and N-hydroxysuccinimide (NHS) are used to increase the crosslink density of the gelatin of the present invention. Then, poly-N-isopropylacrylamide (PIPAAm) is grafted to the cell carrier to obtain a cell carrier with temperature-responsive for being attracted by the magnetic field device and to enhance the stability of the cell carrier at the bottom of the powerless bioreactor.

In a second aspect, the present invention provides a method for recovering a magnetic cell carrier combined with a powerless system, which comprises the following steps:

(a) washing the culturing cell carrier with PBS;

(b) adding a pre-cooled culture medium;

(c) using a hemocytometer for cell count analysis; the temperature of the culture medium is between 10° C. and 20° C., and are treated for 30 minutes.

In a third aspect, the present invention provides a microfluidic device for manufacturing the cell carrier, comprising a continuous phase infusion element, a dispersed phase infusion element, and an iced element, wherein the continuous phase infusion element comprises a microfluidic tube, and the continuous phase infusion element and the microfluidic tube both fill with olive oil; wherein the dispersed phase infusion element comprises an injection needle, and the dispersed phase infusion element was filled with a 10% gelatin aqueous solution, which can be imported to the microfluidic tube through the injection needle; wherein the ice component comprises a container and an ice bucket, and the container is used to contain the liquid from the microfluidic tube and freeze it.

In an embodiment wherein said the dispersed phase infusion element can comprise 0.1 g/mL iron oxide (Fe₃O₄), and the Fe₃O₄ solution (10% w/v) and the gelatin aqueous solution (10% w/v) are mixed at a volume ratio of 1:1.

The magnetic cell carrier combined with a powerless bioreactor system of the present invention can provide culture medium continuously through cell carrier preparation, automatic culture medium replacement, static magnetic field stimulation cell generation, and optimized cell centralized management and cell recovery rate. Therefore, the magnetic cell carrier and biological incubator system of the present invention can improve the efficiency of cell culture, produce a large number of cells and stabilize the quality of cell culture.

### BRIEF DESCRIPTION OF THE DRAWINGS

FIG. 1 shows a schematic diagram of a system with a magnetic bio-carrier combined with a powerless bioreactor system of the present invention.

FIG. 2 shows a schematic diagram of the microfluidic device for manufacturing the biological carriers of the present invention.

FIG. 3 shows an FTIR spectrum of grafted PIPAAm on the cell carrier of the present invention.

FIG. 4 shows the SEM result of the prior art carrier and the cell carrier of the present invention.

FIG. 5 shows the result of the toxicity test of the biological material of the present invention.

FIG. 6 shows the result of the cell proliferation of the system of the present invention.

FIG. 7 shows the result of the cell recovery of the system of the present invention.

FIG. 8 shows genotype expression of the system of the present invention by Q-PCR.

### DETAILED DESCRIPTION OF THE PREFERRED EMBODIMENTS

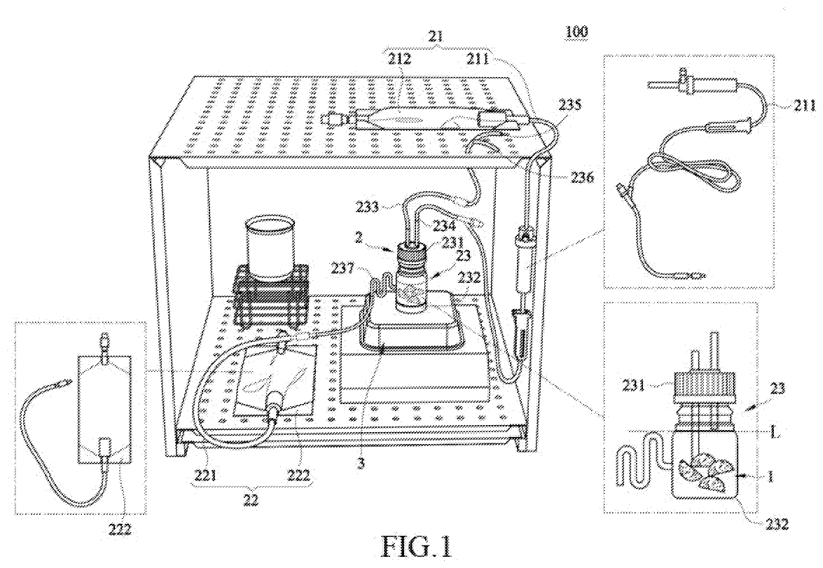

Other objectives, structural composition, application functional characteristics, and effects of the present invention will become more apparent from the following detailed description when taken in conjunction with the accompanying drawings. Referring to FIGS. 1 to 8, the present invention proposes a system 100 combining a magnetic cell carrier with a non-powered bioreactor for culturing cells, comprising a cell carrier 1, a powerless bioreactor 2, and a magnetic field device 3, wherein:

The cell carrier 1 has a plurality of pores on its surface and has non-smooth wrinkles to help the cells culture in the culture medium not slip easily; the interior of the cell carrier 1 is a three-dimensional porous structure with good pore connectivity and a high proportional surface area. The size of the pores of the cell carrier 1 is suitable for cell attachment, growth, and proliferation. The cell carrier 1 mimics the three-dimensional space and physical properties of the extracellular matrix (ECM) network through a three-dimensional culture method. The three-dimensional porous structure of the cell carrier 1 can facilitate cell proliferation, nutrients and metabolites exchange, gas diffusion, growth factors secreted by cells and message transmission functions, and increase the interaction between cells or between cells and ECM. The materials, geometric shapes, and microstructures of the cell carrier 1 and the scaffold are designed to simulate the stable growth environment of the cells in the organism vary based on cell specificity. The diameter of the cell carrier 1 is from 0.01 mm to 20 mm. In the embodiment of the present invention, the diameter of the cell carrier is selected from 8 mm to 9 mm, but the diameter and appearance of the cell carrier 1 are not limited to practical applications.

Preferably, the cell carrier 1 of the present invention is a natural biopolymers gelatin mixed with magnetic nanoparticles, and a chemical cross-linking agent is used to enhance the mechanical properties and strength of the gelatin carrier to meet cells growth requirements, thereby promote cell proliferation or induce cell differentiation into tissues, and with amine groups on gelatin to form stable cross-links.

Preferably, the magnetic nanoparticles used in the cell carrier 1 can be nickel (Ni) nanoparticles, cobalt (Co) nanoparticles, or iron oxides nanoparticles, such as γ-Fe₂O₃ and iron oxide (Fe₃O₄), or complex nanoparticles, such as FePt, CoPt, CoFe₂O₄, MgFe₂O₄, etc. In the embodiment of the present invention, the magnetic nanoparticle is selected from Fe₃O₄, but the composition of the magnetic nanoparticle is not limited to practical applications.

Preferably, the chemical cross-linking agent used in the cell carrier 1 is glutaraldehyde. Glutaraldehyde is relatively easily accessible and low cost. Its aqueous solution can cross-link biological tissue materials in a short period with significant-high efficiency and stability, and therefore can also stably cross-link with the gelatin of the present invention.

Next, another water-soluble cross-linking agent carbodiimide (EDC) and N-hydroxysuccinimide (NHS) are used to increase the crosslink density of the gelatin of the present invention. Then, poly-N-isopropylacrylamide (PIPAAm) is grafted to prepare the cell carrier 1 with temperature-responsive for being attracted by the magnetic field device 3 and to enhance the stability of the cell carrier 1 at the bottom of the powerless bioreactor 2.

Preferably, the PIPAAm polymer used in the cell carrier 1 has a lower critical solution temperature (LCST) to drive through a phase transition from hydrophobic polymer to hydrophilic polymer when the rising temperature. The PIPAAm-grafted cell carrier 1 surface was expected to exhibit temperature-responsive material by phase transition characteristics. When the temperature is lower than LCST, the surface of cell carrier 1 with PIPAAm-grafted is hydrophilic; when the temperature is high than LCST, the grafted surface is hydrophobic. PIPAAm exhibits a reversible temperature-dependent phase in thermally modulated cell conjugate and separation.

In other words, in the present invention, the cells would adhere to the pores of the cell carrier 1 at 37° C. and going cell diffusion and proliferation; the cultured cells on the cell carrier 1 became detached when the culture's temperature was lowered to 20° C. due to PIPAAm's hydration/dehydration alteration.

As shown in FIG. 1, the powerless bioreactor 2 comprises a micro-infusion element 21, a culture fluid collection element 22, and a gooseneck cell culture tank 23.

The micro-infusion element 21 comprises an infusion tube 211 and a supply container 212. The micro-infusion element 21 is responsible for importing fresh culture solution into the gooseneck cell culture tank 23.

The culture fluid collection element 22 comprises a collection tube 221 and a collection container 222. The culture solution collecting element 22 is used for recovering the culture medium containing cell metabolites discharged from the gooseneck cell culture tank 23.

The gooseneck cell culture tank 23 comprises a cover 231 and a bottle 232. The cover 231 comprises a first hole 233 and a second hole 234. The first hole 233 comprises an air outlet tube 235 and an air inlet tube 236, and the air inlet tube 236 is used for supplying oxygen to the cells. The air outlet tube 235 is used to discharge the carbon dioxide released by the cells. The second hole 234 is used for importing fresh culture medium into the gooseneck cell culture tank 23. The bottle 232 comprises a gooseneck tube 237. The gooseneck tube 237 is used for draining the culture medium containing cell metabolites to the culture fluid collection element 22.

Preferably, the internal space of the gooseneck cell culture tank 23 is connecting with the micro-infusion element 21 and the culture fluid collection element 22, and the micro-infusion element 21 can slowly import fresh culture medium through the infusion tube 211 to the gooseneck cell culture tank 23; when the culture medium in the gooseneck cell culture tank 23 is higher than an overflow position L, the medium containing cell metabolites can be automatically discharged through the gooseneck tube 237 to the culture fluid collection element 22 by the principle of communicating vessels to reduces the risk that the culture medium might be contaminated during culture. The nutrient in the gooseneck cell culture tank 23 adopts a semi-replacement condition to simulate the dynamic environment in which nutrients and metabolites coexist in the body. The infusion tube of the micro-infusion element is connecting to the second hole of the gooseneck cell culture tank; the collection tube of the culture fluid collection element is connecting to the gooseneck tube of the gooseneck cell culture tank.

The magnetic field device 3 comprises at least one magnet used for generating a static magnetic field. The magnetic field device 3 is connected to the magnet to provide a fixed magnetic field of 0.1T to attract the cell carrier 1.

The present invention further provides a method for recovering a magnetic cell carrier combined with a powerless system, which comprises the following steps:

(a) washing the culturing cell carrier with PBS

(b) adding a pre-cooled culture medium;

(c) using a hemocytometer for cell count analysis; the temperature of the culture medium is between 10° C. and 20° C., and are treated for 30 minutes.

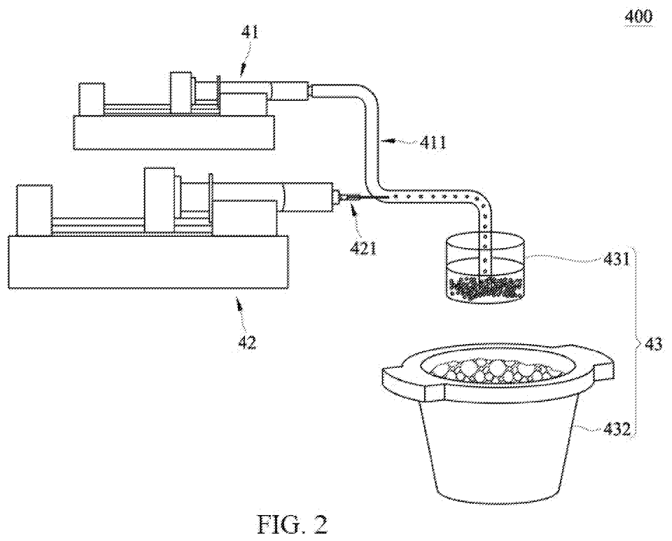

As shown in FIG. 2, the present invention further provides a microfluidic device 400 for manufacturing the cell carrier 1, comprising a continuous phase infusion element 41, a dispersed phase infusion element 42, and an iced element 43.

The continuous phase infusion element 41 comprises a microfluidic tube 411, the continuous phase infusion element 41 and the microfluidic tube 411 are connected, and the continuous phase infusion element 41 and the microfluidic tube 411 are both filled with the olive oil so that the olive oil can flow from the continuous phase infusion element 41 to the microfluidic tube 411.

The dispersed phase infusion element 42 comprises an injection needle 421, and the dispersed phase infusion element 42 is filled with a 10% gelatin aqueous solution. In addition, the injection needle 421 is inserted into the microfluidic tube 411 and then the 10% gelatin aqueous solution can be injected into the microfluidic tube 411. Preferably, the dispersed phase infusion element 42 may comprise a 0.1 g/mL iron oxide (Fe₃O₄) solution. The Fe₃O₄ solution (10% w/v) and the gelatin aqueous solution (10% w/v) are mixed in a volume ratio of 1:1 in the dispersed phase infusion element 42.

The ice component 43 comprises a container 431 and an ice bucket 432. The ice bucket 432 comprises ice cubes, and then the container 431 is placed in it. The container 431 can contain the liquid from the microfluidic tube 411 and freeze the liquid, that is to say, the liquid in the container 431 comprises the olive oil from the continuous phase infusion element 41 and the mix containing Fe₃O₄ solution and gelatin aqueous solution from the dispersed phase infusion element 42.

Furthermore, the method of using the microfluidic device 400 comprises the following steps:

(1) preparing 0.1 g/mL gelatin aqueous solution (10% w/v) with double distilled water as the solvent;

(2) preparing the cell carrier with the microfluidic device. Olive oil is used as the continuous phase and is injected into the microfluidic channel with a flow rate of 500 mL/hr. The dispersed phase is a 10% gelatin aqueous solution, and is injected into the microfluidic channel with a flow rate of 30 mL/hr;

(3) the cell carrier prepared from step (2) is solidified on ice for 15 minutes, and rinsing the cell carrier with acetone several times to remove the olive oil;

(4) cross-linking the cell carrier with 1% glutaraldehyde at room temperature for two hours, and then washing 3 times in deionized water to remove excess glutaraldehyde;

(5) dissolving the cross-linking agent EDC/NHS in 0.1 M MES buffer (pH 6), and adding the cell carrier from step (4), and then rotating at room temperature for 1 day;

(6) adding 27 μM PIPAAm with an amine end group and rotating at room temperature for 1 day to graft PIPAAM on the gelatin carrier;

(7) washing the grafted gelatin carrier of step (6) three times with double distilled water, and freezing at −80° C. overnight. The cell carrier of the present invention can be finally obtained by removing water through a lyophilizer. The porous structure of the cell carrier can be observed by scanning electron microscope (SEM).

Preferably, Fe₃O₄ solution (10% w/v) can be added in a volume ratio of 1:1 in step 1.

As described above, the cell carrier 1 can also be made through the following steps:

(1) preparing 0.1 g/mL iron oxide (Fe₃O₄) solution and 0.1 g/mL gelatin aqueous solution with double distilled as the solvent; mixing the Fe₃O₄ solution (10% w/v) and gelatin aqueous solution (10% w/v) at a volume ratio of 1:1; shaking by an ultrasonic oscillator for 3 hours to evenly disperse Fe₃O₄ nanoparticles. 250 μL mixture is pipetted and dropped on the polytetrafluoroethylene (PTFE) membrane to form a gelatin-Fe₃O₄ gel by placing it on ice. The gelatin-Fe₃O₄ gel is placed in the refrigerator at −20° C. for 1 day and then be freeze-dried.

(2) cross-linking the freeze-dried gelatin-Fe₃O₄ from step (1) with 0.1% glutaraldehyde, and rinsing with water at 25° C. after 2 days later;

(3) dissolving the PIPAAm (27 mM) in 0.1 M MES buffer (pH 6), and then adding to the gelatin-Fe₃O₄ carrier from step (2) to swell for 1 day;

(4) adding the cross-linking agent EDC/NHS into the gelatin-Fe₃O₄ carrier of step (3) to graft PIPAAm onto the gelatin-Fe₃O₄ carrier at 4° C. for 2 days. After the completion of the reaction, the grafted gelatin-Fe₃O₄ carrier is washed with double distilled water twice and frozen at −20° C. for 1 day. The cell carrier 1 of the present invention can be obtained by removing water through the lyophilizer, and the porous structure of the cell carrier 1 could be observed by the scanning electron microscope (SEM).

The cell carrier 1 obtained from the above steps was compared with the prior art cell carrier.

TABLE 1
Abbreviation list of experimental materials of cell carriers

| Name | Abbreviation |
|---|---|
| Gelatin Disk | GD |
| Gelatin Disk - added Fe₃O₄ | Fe₃O₄-GD |
| Gelatin Disk - grafted PIPAAm | P-GD |
| Gelatin Disk - added Fe₃O₄ & grafted PIPAAm | P-Fe₃O₄-GD |

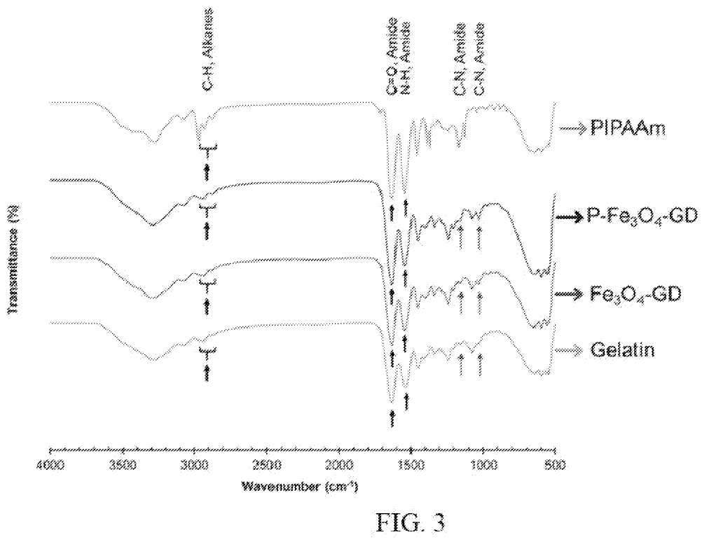

As shown in FIG. 3, Fourier transforms infrared (FTIR) spectroscopy is used to confirm an amide bond formation of the cell carrier 1 of the present invention and grafting detection. A spectra comparison between the GD, PIPAAm, Fe₃O₄-GD, and P—Fe₃O₄-GD showed the peak at 1650 cm⁻¹ indicate an amide I bond, arising from C=0 stretching. The peak at 1530 cm⁻¹ corresponded to an amide II bond, arising from N—H deformation; an amide III bond is C—N stretching.

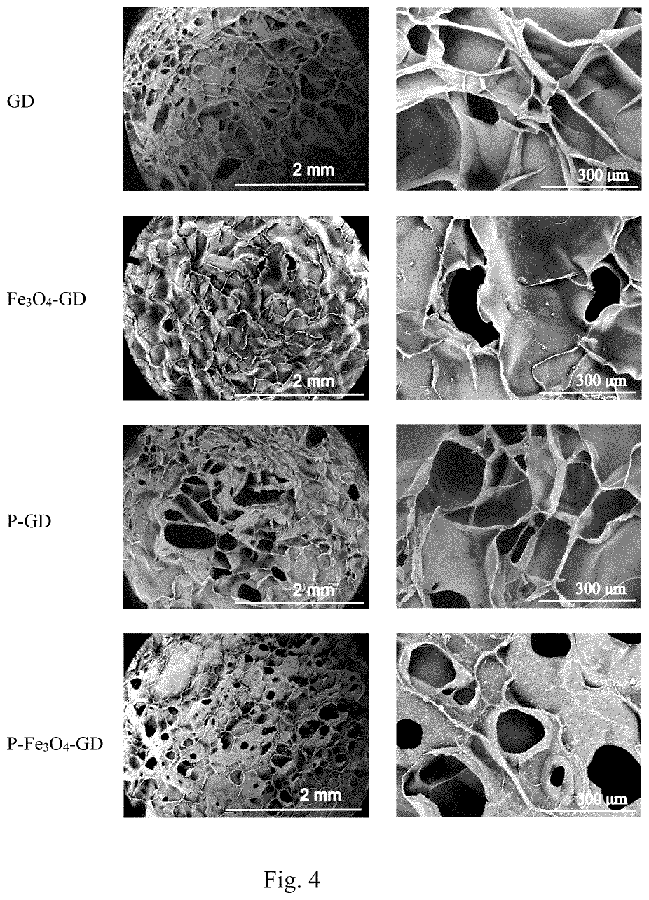

As shown in FIG. 4, the surface morphologies of the cell carrier are observed by scanning electron microscope (SEM). From the observation results, conjugation of PIPAAm onto the P—Fe₃O₄-GD does not affect the surface morphology of the cell carrier; other groups of carriers (GD, Fe₃O₄-GD, PGD) also show non-smooth and wrinkled surface structures.

Cell Line Culture:

The cell line selected for the research material of the present invention is cbMSC-hTERT mesenchymal stem cells derived from human umbilical cord blood, which is purchased from the Bioresource Collection and Research Center of the Food Industry Research Development Institute. Cells were cultured in α-MEM (Minimum Essential Medium Alpha Medium) supplemented with 20% fetal bovine serum (FBS), and 1% antibiotic. The cells were maintained in a cell incubator at 37° C. and 5% CO₂.

Cytotoxicity of the Cell Carrier Assessment

cbMSC-hTERT cells were cultured with the cell carrier of each group on a 96-well plate for 1 day. The water-soluble tetrazolium salt (WST-1) solution was added to test cell proliferation activity.

TABLE 2
Biocompatibility test group

| | Reagent Control | medium |
|---|---|---|
| Control | Positive Control: | 72-hour extract |
| group | | of ZDEC film |
| | Negative Control | 72-hour extract |
| | | of HDPE film |
| Experimental | Gelatin Disk (GD): | 72-hour extract |
| group | | of GD |
| | Grafted PIPAAm Gelatin | 72-hour extract |
| | disk (P-GD): | of PGD |
| | Added Fe3O4 -Gelatin | 72-hour extract |
| | Disk (Fe₃O₄-GD): | of GD-Fe |
| | Grafted PIPAAm added | 72-hour extract |
| | Fe₃O₄ -Gelatin | of P-Fe₃O₄-GD |
| | Disk (P-Fe₃O₄-GD): | |
| Background | Blank: | Cell culture |
| | | medium as the |
| | | background value |

Cell Viability Test WST-1

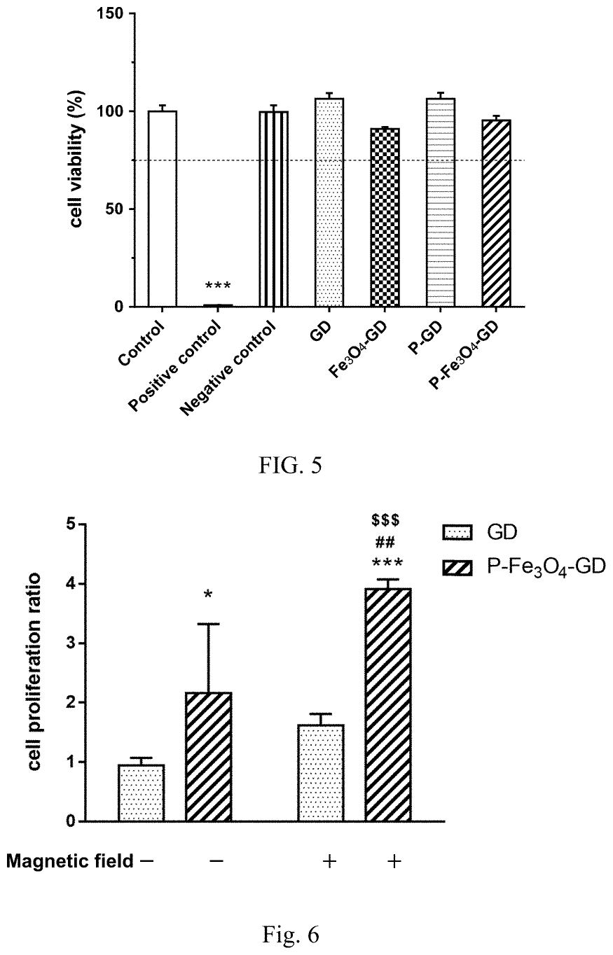

cbMSC-hTERT cells (1×10⁴ cells/well) were cultured on a 96-well plate in a cell incubator at 37° C. and 5% CO₂ for 24 hours. Then, the media was removed, the cells were washed with PBS and separately incubated with 100 μl/well material extract, ZDEC extract, HDPE extract, GD extract, Fe₃O₄-GD extract, P-GD extract, P—Fe3O4-GD extract, and medium only in the incubator for 1 day. After that, the above extract was removed and washed with PBS separately, and then 100 μL of water-soluble tetrazolium salt (WST-1) solution diluted twice with cell culture medium was added to each well and incubated for 1 hour in the dark. Then pipetting 100 μl from each well of the 96-well plate and using an ELISA reader, the absorbance values of each well were measured at 450 nm. As shown in FIG. 5, the results showed that the following formulas are calculated to be higher than 75% of the ISO10993 standard. The results showed that the cell carrier of the present invention was not toxic and has good biocompatibility.

$$Cell viability ( \% ) = \frac{experimental value}{control value} \times 100 \%$$

Cell Analysis

Cell Proliferation in the Magnetic Field Assessment

Cells were seeded on GD and grafted PIPAAm cell carriers (P—Fe₃O₄-GD) separately and cultured with or without 0.1T magnetic field stimulation in the bioreactor for 7 days. The cells were analyzed for viability using Presto Blue viability staining; the culturing efficiency of each group of the cell carriers was detected using the high-sensitivity multi-function micro photon detector (EnSpire) As shown in FIG. 6, the results showed that the group with a magnetic field stimulation in the bioreactors can improve the cell's nutrient transfer and help cell proliferation stably. In the group without the magnetic field, the cells on the cell carrier were removed due to the low density of the cell carrier and then floating above the culture medium and resulting in a large standard deviation. *: p<0.05, **: p<0.01, ***: p<0.001 (compared to GD group); #: p<0.05, ##: p<0.01, ###: p<0.001 (compared to the P—Fe₃O₄-GD group); $: p<0.05, $$: p<0.01, $$: p<0.001 (compared to the GD group with the magnetic field).

Cell Recovery Assessment

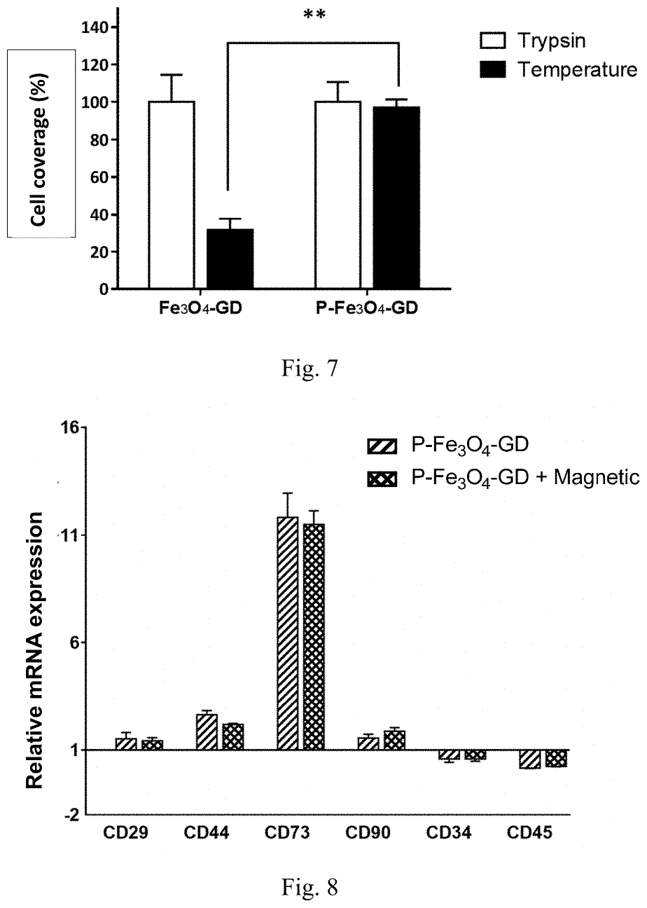

The cells were seeded and cultured on Fe₃O₄-GD and P—Fe₃O₄-GD cell carriers separately for 7 days. The cells were detached by trypsin or lowing temperature respectively. After both treatments for 30 minutes, the number of detached cells was counted with a hemocytometer. As shown in FIG. 7, the results showed that the cell carrier of the culture system of the present invention could also effectively recover cells by cooling. *: p<0.05, **: p<0.01, ***: p<0.001 (compared to Fe3O4-GD group).

Cell Recovery Assessment in the Culture System of the Present Invention by the Real-Time PCR Analysis

The cells were seeded in a 10 cm dish and the cell carrier (P—Fe₃O₄-GD) of the present invention with static magnetic field stimulation for 7 days separately. Q-PCR was used for analyzing gene expression to evaluate whether the phenotype of stem cells will be changed in the present invention for long-term culture; or whether the phenotype of stem cells will be affected by static magnetic field stimulation. According to International Society for Cellular Therapy (ISCT) guidelines, the cell markers of stem cells must express CD73 and CD90 and lack expression of CD34 and CD45. In addition, mesenchymal stem cells (MSC) derived from umbilical cord blood markers well-described in the literature (CD29 and CD44). As shown in FIG. 8, the results showed that the groups cultured in the culture system of the present invention all express CD29, CD44, CD73, and CD90 stem cells, confirming that the cells could still maintain the phenotype of stem cells in the culture system of the present invention for multiple days.

The magnetic cell carrier combined with a powerless bioreactor system of the present invention can provide culture medium continuously through cell carrier preparation, automatic culture medium replacement, static magnetic field stimulation cell generation, and optimized cell centralized management and cell recovery rate. Therefore, the magnetic cell carrier and biological incubator system of the present invention can improve the efficiency of cell culture, produce a large number of cells and stabilize the quality of cell culture.

The foregoing descriptions of various embodiments of the invention are provided for purposes of illustration and are not intended to be exhaustive or limiting. Modifications or variations are also possible in light of the above teachings. All such modifications and variations are within the scope of the invention.

What is claimed is:

1. A magnetic cell carrier combined with a powerless bioreactor system for culturing cells, comprising:
a cell carrier has a porous and wrinkling surface to enhance the ability for cell attachment; the interior of the cell carrier is a three-dimensional porous structure, which enables large surface area and space; the cell carrier also exhibits temperature-responsive cell adhesion/detachment properties
a powerless bioreactor comprising
a micro-infusion element comprises an infusion tube and a supply container, wherein the micro-infusion element is responsible for importing fresh culture solution into a gooseneck cell culture tank;
a culture fluid collection element comprises a collection tube and a collection container, wherein culture fluid collection element is used for recovering the culture medium containing cell metabolites discharged from the gooseneck cell culture tank;
the gooseneck cell culture tank comprises a cover and a bottle, wherein the cover comprises a first hole and a second hole; wherein the first hole comprises an air outlet tube and an air inlet tube; wherein the air inlet tube is used for supplying oxygen to cells, and the air outlet tube is used to discharge the carbon dioxide released by the cells; the second hole is used for importing fresh culture medium into the gooseneck cell culture tank; the bottle comprises a gooseneck tube, which is used for draining the culture medium containing cell metabolites to the culture fluid collection element.
wherein the gooseneck cell culture tank is connecting with the micro-infusion element and the culture fluid collection element, and the micro-infusion element can slowly import fresh culture medium through the infusion tube to the gooseneck cell culture tank; when the culture medium in the gooseneck cell culture tank is higher than an overflow position L, the medium containing cell metabolites can be automatically discharged through the gooseneck tube to the culture fluid collection element by the principle of communicating vessels to reduces the risk that the culture medium might be contaminated during culture; the nutrient in the gooseneck cell culture tank adopted a semi-replacement condition to simulate the dynamic environment in which nutrients and metabolites coexist in the body; and,
a magnetic field device,
wherein the cell carrier is a natural biopolymers gelatin mixed with magnetic nanoparticles, and a chemical cross-linking agent is used to enhance the mechanical properties and strength amino group of the gelatin; another water-soluble cross-linking agent carbodiimide (EDC) and N-hydroxysuccinimide (NHS) are used to increase the crosslink density of the gelatin; then, poly-N-isopropylacrylamide (PIPAAm) is grafted to the cell carrier to obtain cell carrier with temperature-responsive for being attracted by the magnetic field device, and to enhance the stability of the cell carrier at the bottom of the powerless bioreactor.

2. The system according to claim 1, wherein the cell carrier comprises nickel (Ni) nanoparticles, cobalt (Co) nanoparticles, or iron oxides nanoparticles (γ-Fe₂O₃ and Fe₃O₄), or complex nanoparticles, such as FePt, CoPt, CoFe₂O₄, MgFe₂O₄.

3. The system according to claim 1, wherein the diameter of the cell carrier is from 0.01 mm to 20 mm.

4. The system according to claim 1, wherein the chemical cross-linking agent used in the cell carrier is glutaraldehyde.

5. The system according to claim 1, wherein the infusion tube of the micro-infusion element is connecting to the second hole of the gooseneck cell culture tank; the collection tube of the culture fluid collection element is connecting to the gooseneck tube of the gooseneck cell culture tank.

6. The system according to claim 1, wherein the magnetic field device comprises at least one magnet for generating a static magnetic field.

7. A microfluidic device for manufacturing the cell carrier according to claim 1, comprising:
a continuous phase infusion element, comprising a microfluidic tube, wherein the continuous phase infusion element and the microfluidic tube are both fill with olive oil;
a dispersed phase infusion element, comprising an injection needle, wherein the dispersed phase infusion element is filled with a 10% gelatin aqueous solution, and the 10% gelatin aqueous solution is injected into the microfluidic tube through the injection needle; and,
an iced element comprises a container and an ice bucket wherein the container can contain the liquid from the microfluidic tube and freeze the liquid;
wherein dispersed phase infusion element comprises a 0.1 g/mL iron oxides (Fe₃O₄) solution, and the Fe₃O₄ solution (10% w/v) and the gelatin aqueous solution (10% w/v) are mixed in a volume ratio of 1:1.

8. A method for using the microfluidic device according to claim 7, comprising the following steps:
(1) preparing 0.1 g/mL gelatin aqueous solution (10% w/v) with double distilled water as the solvent;
(2) preparing the cell carrier with the microfluidic device; olive oil is used as the continuous phase and is injected into the microfluidic channel with a flow rate of 500 mL/hr; the dispersed phase is a 10% gelatin aqueous solution, and is injected into the microfluidic channel with a flow rate of 30 mL/hr;
(3) the cell carrier prepared from step (2) is solidified on ice for 15 minutes, and rinsing the cell carrier with acetone several times to remove the olive oil;
(4) cross-linking the cell carrier with 1% glutaraldehyde at room temperature for two hours, and then washing 3 times in deionized water to remove excess glutaraldehyde;
(5) dissolving the cross-linking agent EDC/NHS in 0.1 M IVIES buffer (pH 6), and adding the cell carrier from step (4), and then rotating at room temperature for 1 day;
(6) adding 27 μM PIPAAm with an amine end group and rotating at room temperature for 1 day to graft PIPAAM on the gelatin carrier;
(7) washing the grafted gelatin carrier of step (6) three times with double distilled water, and freezing at −80° C. overnight; the cell carrier of the present invention can be finally obtained by removing water through a lyophilizer; the porous structure of the cell carrier can be observed by scanning electron microscope (SEM);
wherein Fe₃O₄ solution (10% w/v) can be added in a volume ratio of 1:1 in step 1.

9. A method for using the microfluidic device according to claim 7, comprising the following steps:
(1) preparing 0.1 g/mL iron oxide (Fe₃O₄) solution and 0.1 g/mL gelatin aqueous solution with double distilled as the solvent; mixing the Fe₃O₄ solution (10% w/v) and gelatin aqueous solution (10% w/v) at a volume ratio of 1:1; shaking by an ultrasonic oscillator for 3 hours to evenly disperse Fe₃O₄ nanoparticles; 250 μL mixture is pipetted and dropped on the polytetrafluoroethylene (PTFE) membrane to form a gelatin-Fe₃O₄ gel by placing on ice; the gelatin-Fe₃O₄ gel is placed in the refrigerator at −20° C. for 1 day and then be freeze-dried;
(2) cross-linking the freeze-dried gelatin-Fe₃O₄ from step (1) with 0.1% glutaraldehyde, and rinsing with water at 25° C. after 2 days later;
(3) dissolving the PIPAAm (27 mM) in 0.1 M MES buffer (pH 6), and then adding to the gelatin-Fe₃O₄ carrier from step (2) to swell for 1 day;
(4) adding the cross-linking agent EDC/NHS into the gelatin-Fe₃O₄ carrier from step (3) to graft PIPAAm onto the gelatin-Fe₃O₄ carrier at 4° C. for 2 days; after the completion of the reaction, the grafted gelatin-Fe₃O₄ carrier is washed with double distilled water twice and frozen at −20° C. for 1 day; the cell carrier can be obtained by removing water through the lyophilizer.

10. A method for recovering from the system according to claim 1 comprising the following steps:
(a) washing the culturing cell carrier with PBS
(b) adding a pre-cooled culture medium; and,
(c) using a hemocytometer for cell count analysis.

11. The method according to claim 10, wherein the temperature of the culture medium is between 10° C. and 20° C., and is treated for 30 minutes.
